(19) United States
(12) Patent Application Publication
(10) Pub. No.: US 2024/0041711 A1
(43) Pub. Date: Feb. 8, 2024

(54) COSMETIC CARE OR FRAGRANCE COMPOSITION IN TWO-PHASE FORM

(71) Applicant: LVMH RECHERCHE, SAINT JEAN DE BRAYE (FR)

(72) Inventors: Elodie CHAMPREDONDE, SAINT JEAN DE BRAYE (FR); Valérie BOUCHARD DE LA POTERIE, SAINT JEAN DE BRAYE (FR)

(21) Appl. No.: 18/258,964

(22) PCT Filed: Dec. 17, 2021
(86) PCT No.: PCT/FR2021/052393

§ 371 Date: Jun. 22, 2023

(30) Foreign Application Priority Data
Dec. 23, 2020 (FR) FR2014092

(51) Int. Cl. A61K 8/03; A61K 8/37; A61K 8/58; A61K 8/98; A61Q 19/00; A61K 8/67; A61Q 13/00

(52) U.S. Cl. CPC A61K 8/03; A61K 8/37; A61K 8/585; A61K 8/987; A61Q 19/00; A61K 8/676; A61Q 13/00; A61K 2800/262; A61K 2800/436

(57) ABSTRACT

The invention relates to a two-phase cosmetic composition comprising an aqueous phase and an oily phase, the aqueous phase representing at least 50% by weight of the total weight of the composition, the oily phase comprising at least one aromatic silicone oil a) and at least one second oil different from the aromatic silicone oil a), and the density of the oily phase being higher than the density of the aqueous phase at 20° C. The invention also relates to a skin care and/or fragrance application process comprising a step of applying a cosmetic composition according to the invention preferably by spraying.

### TECHNICAL FIELD

The present invention relates to a novel two-phase cosmetic composition for care or fragrance having an original visual appearance due to the presence of two distinct phases and excellent application properties such as the absence of tacky feeling to the touch and the ability to be sprayed. The present invention relates more particularly to a care or fragrance composition in two-phase form, as well as to a skin care and/or fragrance application process comprising a step of applying a cosmetic composition according to the invention, preferably by spraying.

The cosmetics industry has always been looking for compositions that produce surprising effects, such as compositions with an original visual appearance, for example by dispersing solid particles of different colours in a continuous transparent phase. Cosmetic compositions in which solid spheroids are dispersed in a liquid phase have also been described in the prior art.

Other compositions of the prior art comprise capsules whose core is liquid and whose outer wall is solid, obtained by a coacervation process.

The two-phase compositions usually have two distinct layers separated by a horizontal interphase, the light phase being located above the heavy phase. The light phase is generally composed of oil and the heavy phase is mainly composed of water. The arrangement of the phases one above the other is due to the difference in density between the two phases, the density of the oils being lower than that of the water (the oily phase therefore sitting in the upper part of the container).

The invention consists of a skin care composition or a fragrance composition having a two-phase form different from those of the prior art in which the heavy (dense) phase is the oily phase. Thus, the oily phase sits in the lower part of the container in the form of one or more liquid beads, thereby giving an original visual appearance to the final cosmetic composition. The cosmetic composition of the invention is therefore in the form of one or more liquid beads dispersed within a liquid phase of different density, the bead(s) being free of external coating, and in particular of external solid coating.

As for conventional two-phase compositions, when the composition of the invention is shaken, the oily phase (or fatty phase) disperses in the form of droplets in the aqueous phase, the mixture returning to its initial form after a few minutes (5 to 10 minutes), with the oily phase in the form of one or more beads in the lower part of the container.

The cosmetic composition of the invention also differs from the two-phase compositions of the prior art in that it is free of polysaccharide gelling agent, and consequently very fluid (not in gel form). The oily and aqueous phases can thus mix very finely, which makes the cosmetic composition of the invention sprayable (applicable by spraying). In addition, unlike the compositions of the prior art based on polysaccharide gelling agent, the cosmetic composition of the invention is reversible and can recover its initial form after stirring.

The aim of the invention is therefore to propose a novel visual appearance for skin care or fragrance compositions. The care or fragrance composition of the invention must be able to carry cosmetic active agents and/or fragrance by spraying, be stable over time, and have a non-greasy and non-sticky feel.

The care or fragrance composition of the invention also has improved sensory performance, in particular an effect of freshness when the aqueous phase present within the composition of the invention evaporates, while providing an emollient or fragrance effect with the oily phase.

In the case of skin care, it has been observed that the composition of the invention leads to a better stabilisation of the active agents present in the composition, said active agents being very little degraded when they are combined with the mixture of oils claimed by the invention.

In the case of fragrance, it has been observed that the composition of the invention makes it possible to obtain alcohol-free formulations having performances equivalent to an alcohol fragrance, the use of alcohol being currently called into question because of risks for certain users of intolerance, allergy, irritation, photosensitisation and safety related to the use of alcohol.

Another advantage of the invention lies in the absence or the very small quantity of surfactant required, the cosmetic composition of the invention being able to contain less than 0.1% by weight of surfactant, in order to preserve the two-phase appearance of the composition observed at rest and good skin tolerance, without degrading the olfactory and organoleptic properties of the composition.

### GENERAL DESCRIPTION OF THE INVENTION

The first subject of the present invention is a two-phase cosmetic composition comprising an aqueous phase and an oily phase, the aqueous phase representing at least 50% by weight of the total weight of the composition, the oily phase comprising at least one aromatic silicone oil a) and at least one second oil different from the aromatic silicone oil a), and the density of the oily phase being higher than the density of the aqueous phase at 20° C.

A simple test to determine whether the density of the oily phase is greater than the density of the aqueous phase at 20° C. consists of mixing the two phases according to the chosen ratio by shaking, and then, after shaking is discontinued, observing the behaviour of the two phases in relation to each other visually at rest (5 to 10 minutes after shaking). If the oily phase is positioned below the aqueous phase in the container containing them, this means that the density of the oily phase is greater than the density of the aqueous phase at 20° C.

Preferably, the cosmetic composition according to the invention has two non-homogeneous phases, preferably an aqueous phase and an oily phase, which are visually distinct at rest. It may be in the two-phase form comprising two visually distinct phases immiscible at rest (preferably an aqueous phase and an oily phase), which mix easily by shaking to allow homogeneous application, and phase out rapidly after shaking is stopped to regain their initial state (reversible character of the composition of the invention). Preferably, the composition has two visually distinct superimposed phases, preferably having a clear interface. Preferably, the continuous phase of the composition is the aqueous phase, the oily phase being dispersed in the form of one or more spherical beads or else in ovoid form. More preferably, one of the phases, preferably both phases, are transparent. Unlike an emulsion, at rest, the oily phase of the invention is not dispersed homogeneously throughout the aqueous phase, but only in the bottom of the container containing the cosmetic composition of the invention. Unlike an emulsion in which the dispersed fatty phase will coalesce over time, in the two-phase cosmetic composition of the invention, the droplets of the oily phase are in a state of equilibrium which will allow them to be maintained in dispersed form (without coalescence) in the bottom of the container over time.

The composition of the invention may comprise less than 0.1% by weight, relative to the total weight of the composition, of an emulsifying agent such as a surfactant or an emulsifying polymer, which may degrade the two-phase appearance of the composition observed at rest and the olfactory and organoleptic properties of the composition. It preferably comprises less than 0.05%, and more preferably less than 0.01%, by weight of an emulsifying agent or of an emulsifying polymer relative to the total weight of the composition. According to a particularly preferred embodiment, the cosmetic composition of the invention is free of an emulsifying agent such as a surfactant or an emulsifying polymer.

In particular, the composition comprises less than 0.1%, preferably less than 0.05%, and even more preferentially less than 0.01%, by weight relative to the total weight of the composition, of one or more nonionic surfactants, of polyoxyalkylene surfactants comprising at least five units chosen from —CH2CH(OH)CH2— and —OCH2CH2—, such as polyoxyethylene compounds and polyoxypropylene compounds. These surfactants include polyoxyalkylene ethers, such as POE (10) cetyl ether, polyoxyalkylene esters, such as PEG-40 hydrogenated castor oil or POE (20) sorbitan monolaurate, condensates of polyoxyethylene alkylphenols, condensation products of ethylene oxide with the reaction product of propylene oxide and ethylene diamine, polyethoxylated alcohols, polysorbates, and dimethicone copolyols. The composition is preferably free of nonionic surfactant. The term ‘emulsifying polymer’ is understood to mean an amphiphilic polymer, that is to say one endowed with at least one hydrophilic part and at least one hydrophobic part. Hydrophilic groups and hydrophobic groups are well known to the person skilled in the art, capable of helping to form and stabilise emulsions. Examples of emulsifying amphiphilic polymers that may be mentioned include polymers and copolymers based on AMPS, crosslinked or non-crosslinked, polyacrylic acid/alkyl acrylate copolymers, in particular acrylate/C10-C30-alkyl acrylate copolymers and mixtures thereof.

The composition of the invention may comprise less than 0.1% by weight relative to the total weight of the composition of a gelling agent, and more particularly of a polysaccharide gelling agent such as xanthan gum, gellan gum, guar gum, alginates and carrageenans, agar-agar, etc. It preferably comprises less than 0.01%, and more preferably less than 0.001%, by weight of a polysaccharide gelling agent relative to the total weight of the composition. According to a particularly preferred embodiment, the cosmetic composition of the invention is free of a polysaccharide gelling agent. In the absence of gelling agent, and more particularly of polysaccharide gelling agent, the cosmetic composition of the invention is fluid, i.e., in non-gelled form, which makes it sprayable (applicable by spraying).

In the cosmetic composition of the invention, the aqueous phase is greatly predominant and may represent at least 50%, preferably at least 60%, more preferentially at least 65%, and even more preferentially at least 70%, by weight of the total weight of the composition, the remainder representing the % by weight of oily phase.

The arrangement of the aqueous and oily phases within the composition of the invention is due to the difference in density between the two phases. Thus, the density of the aqueous phase is advantageously less than or equal to 1.00 g·cm⁻³ at 20° C. The density of the oily phase is advantageously greater than 1.00 g·cm⁻³ at 20° C., and even more advantageously greater than 1.01 g·cm ⁻³ at 20° C. When the oily phase consists of a mixture of oils, its density can be determined using a densimeter, for example a DMA 500 densimeter from Anton Paar.

According to a preferred embodiment, the difference between the density of the aqueous phase and the density of the oily phase is less than or equal to 0.02.

The oily phase preferably represents at most 50%, more preferentially at most 40%, even more preferentially at most 35% and even more preferentially at most 30%, by weight relative to the total weight of the composition, the remainder representing the % by weight of aqueous phase.

The aromatic silicone oil a) preferably has a density greater than 1.00 g·cm⁻³ at 20° C. and more preferentially greater than 1.01 g·cm⁻³ at 20° C.

Advantageously, the aromatic silicone oil a) is a silicone oil having at least one phenyl group, more advantageously having between one and five phenyl groups, and even more advantageously having five phenyl groups.

According to a preferred embodiment, the aromatic silicone oil a) is a silicone oil having at least one phenyl group chosen from diphenyl dimethicone, trimethylsiloxyphenyl dimethicone, phenylpropyldimethylsiloxysilicate, trimethyl pentaphenyl trisiloxane, and mixtures thereof. In a particularly preferred manner, the aromatic silicone oil a) is trimethyl pentaphenyl trisiloxane (CAS number: 3390-61-2 and INCI name: TRIMETHYL PENTAPHENYL TRISILOXANE).

The ratio between the aromatic silicone oil a) and the second oil different from the aromatic silicone oil a) can advantageously vary from 10/90 to 80/20.

The second oil different from the aromatic silicone oil a) present in the composition of the invention is preferably a hydrocarbon oil and/or an aromatic silicone oil b).

The second oil different from the aromatic silicone oil a) is advantageously soluble in the aromatic silicone oil a).

According to a first preferred embodiment, the cosmetic composition of the invention comprises at least one aromatic silicone oil b) different from the aromatic silicone oil a) having at least one phenyl group, preferably having one, two or three phenyl groups, and more preferentially chosen from phenyl trimethicone (CAS number: 218-320-6 and INCI name: PHENYL TRIMETHICONE), diphenylsiloxy phenyl trimethicone (CAS number: 352230-22-9 and INCI name: DIPHENYLSILOXY PHENYL TRIMETHICONE), and mixtures thereof.

Advantageously, when the second oil is an aromatic silicone oil b) different from the aromatic silicone oil a), the ratio between the aromatic silicone oil a) and the second oil different from the aromatic silicone oil a) varies from 10/90 to 50/50, preferably from 15/85 to 40/60, and more preferentially from 15/85 to 30/70. According to this embodiment, the cosmetic composition of the invention is advantageously free of hydrocarbon oil.

When the cosmetic composition of the invention comprises two different aromatic silicone oils a) and b), advantageously the aromatic silicone oil a) has five phenyl groups and the aromatic silicone oil b) has one, two or three phenyl groups.

According to a second preferred embodiment, the cosmetic composition of the invention comprises, as a second oil different from the aromatic silicone oil a), at least one hydrocarbon oil chosen from hydrocarbon oils of vegetable origin. When the cosmetic composition of the invention comprises at least one hydrocarbon oil of vegetable origin, it may advantageously be a mixture of at least two hydrocarbon oils of vegetable origin, preferably chosen from C4 to C24, more preferentially C6 to C12, and even more preferentially C8 to C10 saturated fatty acid triglycerides. Preferably, the hydrocarbon oil of vegetable origin is chosen from jojoba oil (CAS number: 90045-98-0 and INCI name: SIMMONDSIA CHINENSIS SEED OIL), Limnanthes oil (CAS number: 153065-40-8 and INCI name: LIMNANTHES ALBA SEED OIL), a mixture of caprylic/capric triglycerides (CAS number: 73398-61-5 and 65381-09-1 and INCI name: CAPRYLIC/CAPRIC TRIGLYCERIDE), and mixtures thereof. More preferably, the hydrocarbon oil of vegetable origin is chosen from Limnanthes oil (CAS number: 153065-40-8 and INCI name: LIMNANTHES ALBA SEED OIL), a mixture of caprylic/capric triglycerides (CAS number: 73398-61-5 and 65381-09-1 and INCI name: CAPRYLIC/CAPRIC TRIGLYCERIDE), and mixtures thereof. Even more preferably, the hydrocarbon oil of vegetable origin is a mixture of caprylic/capric triglycerides (CAS number: 73398-61-5 and 65381-09-1 and INCI name: CAPRYLIC/CAPRIC TRIGLYCERIDE).

Advantageously, when the second oil is a hydrocarbon oil, the ratio between the aromatic silicone oil a) and the second oil different from the aromatic silicone oil a) varies from 80/20 to 20/80, preferably from 70/30 to 30/70, more preferentially from 65/35 to 35/70, and even more preferentially from 60/40 to 40/60.

According to a particular embodiment, the oily phase comprises at least one nacre and/or at least one hydrophobic pigment (for example a hydrophobic pigment with a pearlescent effect). The nacre and/or the hydrophobic pigment may represent from 0.01% to 5% by weight of the total weight of the composition, and preferably from 0.05% to 2% by weight of the total weight of the composition.

The cosmetic composition of the invention is in the form of two phases (two-phase composition), an aqueous phase and an oily phase, visually distinct from each other. According to an original aspect of the invention, the oily phase may be in the form of bead(s) immiscible within the aqueous phase. The oily-phase bead(s) are not solid capsules. The oily-phase bead(s) are free of external coating.

The present invention also has for a subject the use of a cosmetic composition according to the invention for skin care and/or as a fragrance.

Another, more specific, subject of the invention concerns a composition for skin care comprising a cosmetic composition according to the invention and a cosmetic active agent. Said cosmetic active agent is advantageously contained both in the aqueous phase and in the oily phase. The cosmetic active agents may be advantageously chosen, for example, from anti-aging active agents, moisturising agents, active agents with a lightening effect, or any other cosmetically-active agent whose addition to the composition of the invention makes it possible to obtain a beneficial effect for the skin. Preferably, the cosmetic active agent may be retinol palmitate, Lipochroman®-6 (dimethylmethoxy chromanol, CAS number: 83923-51-7), or an esterified derivative of vitamin C such as ascorbyl tetraisopalmitate. Even more preferably, the cosmetic active agent is an esterified derivative of vitamin C such as ascorbyl tetraisopalmitate.

Another, more specific, subject of the invention concerns a fragrance composition comprising a cosmetic composition according to the invention and a fragrance concentrate, said fragrance concentrate advantageously being contained in the oily phase.

The term ‘fragrance composition’ is understood to mean a product in liquid form intended to perfume an individual after it has been sprayed or applied to the skin, hair or clothing. Such a product is not rinsed after application.

The composition contains a fragrance concentrate, also known as a ‘perfuming concentrate’. The fragrance concentrate may be chosen, for example, from compounds whose INCI name appears on the list of ingredients of a fragrance composition offered for sale under the name ‘fragrance’. A fragrance concentrate is a compound or mixture of compounds that are at least partially volatile at room temperature, the odour of which is detected. The fragrance concentrate consisting of essential oils must necessarily be diluted to express all its olfactory potential, i.e., a perception evolving over the day after application to the skin, dictated by the presence of odorous organic compounds having volatilities very different from each other. The development of this fragrance concentrate aims to give the fragrance composition a top note, a heart note and a base note. The fragrance concentrate preferably comprises predominantly top notes and heart notes.

The fragrance concentrate is prepared from natural or synthetic organic fragrance materials.

Fragrance materials of natural origin include, for example, extracts of flowers, stems and leaves, of fruits, of fruit bark, of roots, of wood, herbs and grasses, resins and balms.

Moreover, essential oils can also be used, such as, for example, essences of bergamot, rose, lavender, sandalwood, cardamon, sage, chamomile, cloves, lemon balm, mint, leaves of cinnamon, juniper, vetiver, olive, galbanum and labdanum.

Fragrance materials of synthetic origin include, for example, Hedione, ethylene brassylate and Habanolide, benzyl acetate, benzyl benzoate, phenoxyethyl isobutyrate, p-tert-butylcyclohexyl acetate, citronellyl acetate, citronellyl formate, geranyl acetate, linalyl acetate, dimethyl benzyl carbinyl acetate, phenylethyl acetate, linalyl benzoate, benzyl formate, ethyl methylphenylglycinate, alkyl cyclohexyl propionate, styralyl propionate and benzyl salicylate, benzyl ethyl ether, linear alkanals with 8 to 18 carbon atoms, citral, citronellal, citronellyloxyacetaldehyde, cyclamen aldehyde, hydroxycitronellal, ionones such as alpha-isomethyl ionone, and methyl cedryl ketone, anethole, citronellol, eugenol, isoeugenol, geraniol, linalool, phenylethyl alcohol, terpineol and terpenes. These compounds are often in the form of a mixture of two or more of these odorous substances.

Fragrance materials of synthetic origin include, for example, Hedione, ethylene brassylate and Habanolide, benzyl acetate, benzyl benzoate, phenoxyethyl isobutyrate, p-tert-butylcyclohexyl acetate, citronellyl acetate, citronellyl formate, geranyl acetate, linalyl acetate, dimethyl benzyl carbinyl acetate, phenylethyl acetate, linalyl benzoate, benzyl formate, ethyl methylphenylglycinate, alkyl cyclohexyl propionate, styralyl propionate and benzyl salicylate, benzyl ethyl ether, linear alkanals with 8 to 18 carbon atoms, citral, citronellal, citronellyloxyacetaldehyde, cyclamen aldehyde, hydroxycitronellal, ionones such as alpha-isomethyl ionone, and methyl cedryl ketone, anethole, citronellol, eugenol, isoeugenol, geraniol, linalool, phenylethyl alcohol, terpineol and terpenes. These compounds are often in the form of a mixture of two or more of these odorous substances.

Olfactory notes are often classified into families that allow a consumer to know what perception they should expect. Examples include citrus fragrances, aromatics, floral fragrances, musks, fruity fragrances, spices, oriental fragrances, marine fragrances, aquatic notes, chypre fragrances, woody fragrances, ferns, leathers and mixtures thereof

The fragrance concentrate represents at least 1%, preferably at least 3%, more preferentially at least 5%, even more preferentially at least 7% by mass, and better still at least 10%, and even better still at least 15%, by weight relative to the total weight of the composition.

The fragrance concentrate may represent from 3 to 40%, preferably from 5 to 35%, for example from 7 to 30% or from 10 to 25%, by weight relative to the total weight of the composition.

In addition to the ingredients described above, the composition may contain at least one additional cosmetically-acceptable ingredient, such as colouring agents or antioxidants.

These additional ingredients must be neutral to the olfactory sense so as not to cause a change of colour or the appearance of a haze related to stability problems, or to alter the odour of the composition of the invention.

The colouring agents may be either water-soluble or fat-soluble, depending on the phase which it is desired to colour. Examples of colouring agents are: Caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (Cl 15850, Cl 75470), Ext. Violet 2, Red 6-7, Ferric Ferrocyanide, Ultramarines, Acid Yellow 3/Yellow 10, Acid Blue 3 and Yellow 10. Examples of fat-soluble colouring agents are Sudan Red, D&C Red 17, D&C Green 6, beta-carotene, soybean oil, Sudan Brown, D&C Yellow 11, D&C Violet 2, D&C Orange 5, quinoline yellow and annatto. The colouring agents generally represent from 0.01% to 1%, preferably from 0.05% to 0.5%, by weight relative to the total weight of the cosmetic composition.

Antioxidants include, for example, ascorbic acid, di-tert-butyl-p-hydroxytoluene (also called BHT or 2,6-di-tert-butyl-p-cresol), BHA (tert-butyl-4-hydroxyanisole), tocopherols such as vitamin E, tocopherol derivatives such as tocopheryl acetate and gallic acid and its derivatives.

The cosmetic composition of the invention may be packaged in a container optionally provided with an application means. The application means may be a spraying means.

The subject of the invention is also a vial provided with an application means and a packaging means, and containing the composition described previously. The term ‘vial’ is also understood to mean containers of the bottle, ampoule, pipette, etc. type. The packaging means is preferably transparent in order to allow the cosmetic composition of the invention to be perceived, which is preferably itself transparent. The application means may be a manual pump, and preferably a sprayer. The cosmetic composition of the invention can be applied in the form of fine droplets by means of a pressurising device. These devices are well known to the person skilled in the art and include non-aerosol pumps or ‘atomisers’, aerosol containers comprising a propellant, as well as aerosol pumps using compressed air as propellant.

Finally, the subject of the invention is a skin care and/or fragrance application process comprising the following steps:
i—shaking of a cosmetic composition according to the invention, preferably contained in a vial, to mix the aqueous phase with the oily phase, and
ii—application of the mixed cosmetic composition obtained at the end of step i—either directly using the fingers or with a cotton wool, or by spraying, and preferably by spraying.

This process may be a process for the care of the skin or a process for applying fragrance to the skin or hair of an individual, which consists of applying the composition of the invention described above to the skin or hair of the individual, preferably by means of a spraying means. The cosmetic composition of the invention is preferably applied directly to the skin, and more preferentially to the face or to a part of the body which is not the face.

In addition to the above arrangements, the invention also comprises other arrangements which will emerge from the additional description which follows, which relates to the preparation of cosmetic compositions according to the invention.

### EXAMPLES

### Example 1: Evaluation of Different Aromatic Silicone Oils

The behaviour in water of the different aromatic silicone oils presented in Table 1 below was studied at an aqueous phase/oily phase ratio of 90/10:

TABLE 1

| Tradename | INCI name | Density (g · cm⁻³) at 20° C. |
|---|---|---|
| Silshine 151 | Phenylpropyldimethylsiloxysilicate | 1.02 |
| KF-54 HV | Diphenyl dimethicone | 1.07-1.11 |
| KF-56A | Diphenylsiloxy phenyl trimethicone | 0.99-1.005 |
| Silshine 150 | Diphenyl dimethicone | 1.02 |
| DOW CORNING PH-1555 HRI | Trimethyl pentaphenyl trisiloxane | 1.092-1.099 |
| PTM 30cst | Phenyl trimethicone | 0.99 |
| Belsil PDM-1000 | Trimethylsiloxyphenyl dimethicone | 1.07 |
| Belsil PDM-20 | Trimethylsiloxyphenyl dimethicone | >1 |

The different aromatic silicone oils in the aqueous phase are in the following form:
For Silshine 151: Two-phase at rest with a pale yellow flattened bead in the bottom of the vial;
For KF-54 HV: Two-phase at rest with a transparent flattened bead that sticks to the glass walls;
For KF-56A: Positioning of the oil on the surface with a clean and concave interface (no bead formation);
For Silshine 150: Two-phase at rest with a transparent bead in the bottom of the vial;
For DOW CORNING PH-1555 HRI: Two-phase at rest with a very flattened transparent bead in the bottom of the vial;
For PTM 30 cst: Positioning of the oil on the surface with a clean and concave interface (no bead formation);
For Belsil PDM-1000: Two-phase at rest with an opalescent flattened bead in the bottom of the vial; and
For Belsil PDM-20: Two-phase at rest with several transparent beads in the bottom of the vial.

The behaviour of several mixtures of aromatic silicone oils in the aqueous phase was also evaluated at different ratios:
Dow CORNING PH-1555 HRI/PTM 30cst mixture at ratios 10/90, 20/80, 30/70, and 50/50: Two-phase at rest with a transparent bead in the bottom of the vial (beads increasingly flattened as the amount of PTM 30cst is increased); and
Dow CORNING PH-1555 HRI/KF-56A mixture at ratios 10/90, 20/80, 30/70, 40/60 and 50/50: Two-phase at rest with a transparent bead in the bottom of the vial (beads increasingly flattened as the amount of KF-56A is increased).

### Example 2: Test of Substitution of Part of the Aromatic Silicone Oil

In order to meet a growing demand for naturalness (use of ingredients of natural origin), a test was carried out to reduce a portion of the quantity of aromatic silicone oil in a cosmetic formula according to the invention while maintaining a visual appearance in the form of beads; the presence of aromatic silicone oil is actually essential to obtain this original visual appearance.

A formula A comprising 90% by weight of aqueous phase and 10% by weight of oily phase was prepared. The density of formula A is measured using a DMA 500 densimeter from Anton Paar, at a temperature of 22° C. The oily phase consists of the oil mixtures shown in the following Table 2 (% by weight):

TABLE 2

| | DOW CORNING | | Density |
|---|---|---|---|
| | PH-1555 | | (g · cm⁻³) at |
| | HRI | Substitute oil | 20° C. |
| Reference | 10% | — | 1.092-1.099 |
| oil | | | |
| Formula A | 5% | 5% Caprylic/Capric | 1.0155 |
| | | Triglyceride | |

### Observations of Formula A:

In formula A, the replacement of part of the DOW CORNING PH-1555 HRI oil by Caprylic/Capric Triglyceride oil resulted in a composition with satisfactory visual appearance: the fatty phase remains transparent and homogeneous during the step of mixing the two oils, then is positioned in the form of beads in the bottom of the container.

### Example 3: Compatible with Hydrophobic Nacre and Pigments

Formula A of Example 2 was tested in the presence of different nacre particles and a hydrophobic pigment in order to evaluate the affinity of the solid particles with the aqueous and fatty phases and their impact on the stabilisation of the fatty phase in the form of beads.

Formulas A-1 to A-4 of Table 3 were prepared (% by weight):

TABLE 3

| | | Formula | Formula | Formula | Formula |
|---|---|---|---|---|---|
| Phases | Ingredients | A-1 | A-2 | A-3 | A-4 |
| A | Water (aqueous phase) | 89.5 | 89.5 | 89.5 | 89.9 |
| B | DOW CORNING PH-1555 HRI (oily phase) | 5.0 | 5.0 | 5.0 | 5.0 |
| B | Caprylic/Capric Triglyceride (oily phase) | 5.0 | 5.0 | 5.0 | 5.0 |
| C | Hydrophilic nacre with silica (Synthetic | 0.5 | — | — | — |
| | Fluorphlogopite and Titanium Dioxide and Silica | | | | |
| | and Tin Oxide) | | | | |
| C | Silica-free hydrophilic nacre (Synthetic | — | 0.5 | — | — |
| | Fluorphlogopite and Titanium Dioxide and Tin | | | | |
| | Oxide) | | | | |
| C | Hydrophobic treated pearlescent pigment | — | — | 0.5 | 0.1 |
| | (Titanium Dioxide and Mica and | | | | |
| | Triethoxycaprylylsilane) | | | | |

Phase A is the aqueous phase. It is homogenised with stirring.
Phase B is the oily phase. It is pre-dispersed separately.
Phase C is the pulverulent phase. It is dispersed in phase B, then the mixture of phases B and C is added to phase A.

The mixture of A, B and C is stirred and then left to rest at room temperature. Observations of the different formulas:
Formula A-1: The hydrophilic nacre with silica stabilises the oily phase into several medium-sized pearlescent beads in the bottom of the container.
Formula A-2: The silica-free hydrophilic nacre similarly stabilises the oily phase into several medium-sized pearlescent beads in the bottom of the container. A slight yellowing is observed.
Formula A-3: The hydrophobically treated pearlescent pigment particles are fully wetted by the oily phase, which remains in the form of a single pearlescent bead. The pigment particles are deposited at the internal interphase of the bead formed by the oily phase, and protect this phase from the aqueous phase. An excess of pearlescent pigment is observed at the bottom of the container.
Formula A-4: A formula similar to formula A-3 is prepared with a smaller amount of hydrophobic treated pearlescent pigment. A single pearlescent bead is obtained, all the pigment particles being arranged at the interphase of the aqueous and oily phases, without any deposit in the bottom of the container.

### Example 4: Formulation of a Care Composition with Several Iridescent Beads

A care formula according to the invention with several iridescent beads corresponding to the composition of Table 4 was prepared:

TABLE 4

| Phase | Tradename | INCI name | % |
|---|---|---|---|
| A | PURIFIED WATER | Aqua | qs 100 |
| A | SODIUM CHLORIDE | Sodium chloride | 0.50 |
| A | VEGETABLE GLYCEROL | Glycerin | 3.00 |
| A | — | Potassium sorbate | 0.24 |
| A | CITRIC ACID | Citric acid | 0.005 |
| | MONOHYDRATE | | |
| A | TRISODIUM CITRATE | Sodium benzoate | 0.12 |
| | 2H₂O | | |
| A | 96.2% VOL ALCOHOL | Alcohol | 3.00 |
| A | PURIFIED WATER | Aqua | 1.00 |
| A | OLIGO HA | Hyaluronic acid | 0.01 |
| B | DOW CORNING PH-1555 | Trimethyl pentaphenyl trisiloxane | 2.00 |
| | HRI | | |
| B | KF-56A | Diphenylsiloxy Phenyl Trimethicone | 8.00 |
| C | CONTEXTURAL 2863 | Synthetic fluorphlogopite (and) titanium dioxide (and) | 0.10 |
| | | silica (and) tin oxide | |
| TOTAL | | | 100 |

Phase A is the aqueous phase. It is homogenised with stirring, ensuring the good dissolution of salts and preservatives.
Phase B is the oily phase. It is pre-dispersed separately.
Phase C is the pulverulent phase. It is dispersed in phase B.
The mixture of phases B and C is added to phase A.
The mixture of A, B and C is stirred and then left to rest at room temperature.

The care composition obtained is packaged in a vial equipped with a spray device. At rest, the composition is in the form of a two-phase composition, the two transparent immiscible phases of which are visually distinct, the oily phase consisting of several iridescent beads being located at the bottom of the vial.

After manual shaking in order to homogenise the composition, the care composition is sprayed onto the skin.

Once the application is complete, the composition is left to rest in its packaging for a few minutes. The formation of several beads at the bottom of the vial is rapidly observed again.

The reversibility of the two-phase composition of the invention to return to its initial state is particularly surprising, especially since it is accompanied for the user by a fresh and pleasant effect on the skin.

### Hydration Test:

The cosmetic composition thus prepared is also tested to study its moisturising properties on the skin.

Hydration is measured by corneometry, using a CORNEOMETER CM825 device supplied by Courage & Khazaka SN ‘NUM_SERIE’ known to the person skilled in the art.

The method of measuring the hydration of the stratum corneum is based on the creation of an electric field on the surface of the skin and the detection, using electrodes, of the variations of the dielectric constant induced by the state of hydration of the upper layer of the epidermis (on this subject, see the article by E. Berardesca, EEMCO guidance for the assessment of stratum corneum hydration: electrical methods—Skin Research and Technology 1997; 3: 126-132).

The cosmetic composition prepared is packaged in a transparent vial equipped with a spray device.

The vial is shaken manually so as to homogenise the two-phase composition, and the composition is sprayed onto the forearm of a volunteer. The amount applied corresponds to 2 μL/cm² of skin. The application area is then protected from friction by placing a metal ring surrounding the application area, fixed to the forearm using an adhesive tape.

A measurement is carried out on the area of application, before application of the cosmetic composition, then 6 hours after application of the cosmetic composition, allowing a period of 15 minutes of stabilisation to elapse after removal of the anti-friction protection, before carrying out the measurement.

The residual film of the cosmetic composition of the invention is wiped from the application area.

The variation in hydration between the measurements at T0 and T+6 h is measured according to the method described above. The measured variation is as follows:
Hydration at T+6 h=+29%.

This result demonstrates a significant moisturising effect of the composition of the invention, 6 hours after application. The cosmetic composition of the invention is therefore particularly advantageous for skin care.

In addition to the markedly improved degree of hydration, the cosmetic composition of Example 4 leads to an effect of freshness and lightness after spraying on the skin.

### Example 5: Formulation of a Care Composition with a Single Transparent Bead

A care formula according to the invention with a single transparent bead corresponding to the composition of Table 5 was prepared:

TABLE 5

| Phase | Tradename | INCI name | % by weight |
|---|---|---|---|
| A | PURIFIED WATER | Aqua | qs 100 |
| A | SODIUM CHLORIDE | Sodium chloride | 0.50 |
| A | VEGETABLE GLYCEROL | Glycerin | 3.00 |
| A | CHLORPHENESIN | Chlorphenesin | 0.27 |
| A | CITRIC ACID MONOHYDRATE | Citric acid | 0.002 |
| A | TRISODIUM CITRATE 2H₂O | Sodium citrate | 0.08 |
| A | 96.2% VOL ALCOHOL | Alcohol | 3.00 |
| A | PURIFIED WATER | Aqua | 1.00 |
| A | OLIGO HA | Hyaluronic acid | 0.01 |
| B | DOW CORNING PH-1555 HRI | Trimethyl pentaphenyl trisiloxane | 2.00 |
| B | KF-56A | Diphenylsiloxy Phenyl Trimethicone | 8.00 |
| TOTAL | | | 100 |

Phase A is the aqueous phase. It is homogenised with stirring.
Phase B is the oily phase. It is pre-dispersed separately.
Phase B is then added to phase A, then the mixture is stirred and allowed to rest at room temperature.

The care composition obtained is packaged in a vial equipped with a spray device. At rest, the composition is in the form of a two-phase composition, the two transparent immiscible phases of which are visually distinct, the oily phase consisting of a single transparent bead located at the bottom of the vial.

After manual shaking in order to homogenise the composition, the care composition is sprayed onto the skin.

As in the preceding example, the cosmetic composition of Example 5 is sprayed onto the skin in the form of fine droplets. The application of the cosmetic composition to the skin produces a feeling of freshness and lightness, while leaving on the skin a film comprising the active agents for skin care.

After resting for a few minutes, the cosmetic composition of the invention returns to the initial state with the oily phase in the form of beads in the bottom of the vial.

Example 6: Formulation of a care composition with a single pearlescent bead. A care formula according to the invention with a single pearlescent bead corresponding to the composition of Table 6 was prepared:

TABLE 6

| | | | % by |
|---|---|---|---|
| Phase | Tradename | INCI name | weight |
| A | PURIFIED WATER | Aqua | qs 100 |
| A | SODIUM CHLORIDE | Sodium chloride | 0.50 |
| A | VEGETABLE GLYCEROL | Glycerin | 3.00 |
| A | CHLORPHENESIN | Chlorphenesin | 0.27 |
| A | CITRIC ACID | Citric acid | 0.002 |
| | MONOHYDRATE | | |
| A | TRISODIUM CITRATE | Sodium citrate | 0.08 |
| | 2H₂O | | |
| A | 96.2% VOL ALCOHOL | Alcohol | 3.00 |
| A | PURIFIED WATER | Aqua | 1.00 |
| A | OLIGO HA | Hyaluronic acid | 0.01 |
| B | DOW CORNING PH-1555 | Trimethyl pentaphenyl | 2.00 |
| | HRI | trisiloxane | |
| B | KF-56A | Diphenylsiloxy Phenyl | 8.00 |
| | | Trimethicone | |
| C | COVAPEARL RED AS | Titanium dioxide (and) | 0.10 |
| | | Mica (and) | |
| | | Triethoxycaprylylsilane | |
| TOTAL | | | 100 |

Phase A is the aqueous phase. It is homogenised with stirring.
Phase B is the oily phase. It is pre-dispersed separately.
Phase C is the pulverulent phase. It is dispersed in phase B.
The mixture of phases B and C is added to phase A.
The mixture of A, B and C is stirred and then left to rest at room temperature.

The care composition obtained is packaged in a vial equipped with a spray device. At rest, the composition is in the form of a two-phase composition, the two transparent immiscible phases of which are visually distinct, the oily phase consisting of a single pearlescent bead located at the bottom of the vial.

After manual shaking to homogenise the composition, the care composition is sprayed onto the skin of the face, to obtain a moisturising effect coupled with a freshness effect upon application.

As in the preceding examples, the cosmetic composition in two-phase form is reformed after a few minutes after use, and is visually in the form of a pearlescent bead at the bottom of the vial filled with a continuous aqueous phase.

### Example 7: Formulation of a Composition Comprising an Aromatic Silicone Oil and a Hydrocarbon Oil

A care formula according to the invention in which part of the aromatic silicone oil has been replaced by a hydrocarbon oil, in this case a caprylic/capric triglyceride oil, and corresponding to the composition of Table 7 has been prepared:

TABLE 7

| Phase | Tradename | INCI name | % |
|---|---|---|---|
| A | PURIFIED WATER | Aqua | qs 100 |
| A | SODIUM CHLORIDE | Sodium chloride | 0.50 |
| A | VEGETABLE GLYCEROL | Glycerin | 3.00 |
| A | CITRIC ACID | Citric acid | <0.0 |
| | MONOHYDRATE | | |
| A | — | Sodium benzoate | 0.10 |
| A | — | Potassium sorbate | 0.20 |
| A | OLIGO HA | Hyaluronic acid | 0.20 |
| B | DOW CORNING PH-1555 | Trimethyl pentaphenyl trisiloxane | 5.00 |
| | HRI | | |
| B | — | Caprylic/Capric Triglyceride | 5.00 |
| C | CONTEXTURAL 2863 | Synthetic fluorphlogopite (and) titanium dioxide | 0.10 |
| | | (and) silica (and) tin oxide | |
| TOTAL | | | 100 |

Phase A is the aqueous phase. It is homogenised with stirring.
Phase B is the oily phase. It is pre-dispersed separately.
Phase C is the pulverulent phase. It is dispersed in phase B.
The mixture of phases B and C is added to phase A.
The mixture of A, B and C is stirred and then left to rest at room temperature.

The cosmetic composition obtained is in two-phase form with the oily phase in the form of beads in the bottom of the vial and the aqueous phase above it, the composition of Example 8 also being applicable by spraying. This example shows that part of the aromatic silicone oil can be replaced by a hydrocarbon oil, while providing the same original visual appearance and ease of application by spraying.

### Example 8: Formulation of Compositions Comprising a Sensitive Active Agent

Two cosmetic compositions according to the invention comprising an active agent sensitive to air or water, in which media the active agents generally degrade rapidly, have been prepared. The active agent tested is an ascorbic acid derivative, i.e., ascorbyl tetraisopalmitate (NIKKOL® VC IP).

The active agent was added to the oily phase of the cosmetic compositions according to the invention, as well as to control compositions free of aromatic silicone oil a) and of a second oil different from aromatic silicone oil a), in the proportions (% by weight) indicated in Tables 8 and 9 below:

TABLE 8

| Phase | Tradename | INCI name | % |
|---|---|---|---|
| A | PURIFIED WATER | Aqua | qs 100 |
| A | RONACARE | Benzyl alcohol | 0.50 |
| A | SYMSAVE H | Hydroxyacetophenone | 0.30 |
| A | NIKKOL ® VC IP | Ascorbyl tetraisopalmitate | 3.50 |
| B | DOW CORNING | Trimethyl pentaphenyl | 8.00 |
| | PH-1555 HRI | trisiloxane | |
| B | — | Caprylic/Capric Triglyceride | 4.50 |
| TOTAL | | | 100 |

The control composition of the composition of Table 8 is identical but without oil (absence of Trimethyl Pentaphenyl Trisiloxane and Caprylic/Capric Triglyceride).

TABLE 9

| | Composition of | Control |
|---|---|---|
| | the invention | composition |
| Aromatic silicone oil a) | 15 | — |
| (DOW CORNING PH 1555 HRI | | |
| Trimethyl Pentaphenyl Trisiloxane) | | |
| Aromatic silicone oil b) | 5 | — |
| (KF-56A Diphenylsiloxy Phenyl | | |
| Trimethicone) | | |

| | |
|---|---|
| Ascorbyl tetraisopalmitate | 15 |
| (NIKKOL ® VC IP) | |
| Preservatives | 1.5 |
| Water | qs 100 |

The cosmetic compositions according to the invention and the control compositions were then kept in an oven at 40° C. for 15 days.

The assay of the active ascorbyl tetraisopalmitate in the compositions was carried out at T0 and T+15 days.

The results obtained for the composition of Table 8 and the corresponding control composition are as follows:
at T0: 3.5% by weight for the composition of the invention and 2.9% by weight for the control composition; and
at T+15 days: 3.17% by weight for the composition of the invention and 0.32% by weight for the control composition.

The results obtained for the compositions of Table 9 are as follows:
at T0: 15.0% by weight for the composition of the invention and 14.2% by weight for the control composition; and
at T+15 days: 14.7% by weight for the composition of the invention and 5.2% by weight for the control composition.

The assay results show a strong degradation of the active agent in the control composition free of aromatic silicone oil.

In the cosmetic composition according to the invention, the active agent dissolved in the oily phase is located at the bottom of the vial and protected from contact with water and air, which prevents any degradation of the active agent and improves its stability.

1. A two-phase cosmetic composition comprising an aqueous phase and an oily phase, the aqueous phase representing at least 50% by weight of the total weight of the composition, characterised in that:
the oily phase comprises at least one aromatic silicone oil a) and at least one second oil different from the aromatic silicone oil a); and
the density of the oily phase is greater than the density of the aqueous phase at 20° C.

2. The composition according to claim 1, characterised in that it comprises less than 0.1%, preferably less than 0.05%, by weight of a surfactant or of an emulsifying polymer, relative to the total weight of the composition, and more preferentially is free of surfactant.

3. The composition according to claim 1, characterised in that it comprises less than 0.1%, preferably less than 0.01%, more preferentially less than 0.001% by weight of a polysaccharide gelling agent, relative to the total weight of the composition, and more preferentially is free of polysaccharide gelling agent.

4. The composition according to claim 1, characterised in that the aqueous phase represents at least 60%, preferably at least 65% and more preferentially at least 70%, by weight relative to the total weight of the composition.

5. The composition according to claim 1, characterised in that the density of the aqueous phase is less than or equal to 1.00 g·cm⁻³ at 20° C.

6. The composition according to claim 1, characterised in that the density of the oily phase is greater than 1.00 g·cm⁻³ at 20° C., and preferably greater than 1.01 g·cm⁻³ at 20° C.

7. The composition according to claim 1, characterised in that the difference between the density of the aqueous phase and the density of the oily phase is less than or equal to 0.02.

8. The composition according to claim 1, characterised in that the oily phase represents at most 50%, preferably at most 40%, more preferentially at most 35% and even more preferentially at most 30%, by weight relative to the total weight of the composition.

9. The composition according to claim 1, characterised in that the aromatic silicone oil a) has a density greater 1.00 g·cm³ at 20° C., and preferably greater than 1.01 g·cm⁻³ at 20° C.

10. The composition according to claim 1, characterised in that the aromatic silicone oil a) is a silicone oil having at least one phenyl group, preferably having between one and five phenyl groups, and even more preferentially having five phenyl groups.

11. The composition according to claim 1 characterised in that the aromatic silicone oil a) is a silicone oil having at least one phenyl group chosen from diphenyl dimethicone, trimethylsiloxyphenyl dimethicone, phenylpropyldimethylsiloxysilicate, trimethyl pentaphenyl trisiloxane, and mixtures thereof, and more preferentially trimethyl pentaphenyl trisiloxane.

12. The composition according to claim 1, characterised in that the second oil different from the aromatic silicone oil a) is a hydrocarbon oil and/or an aromatic silicone oil b).

13. The composition according to claim 1, characterised in that the second oil different from the aromatic silicone oil a) is an aromatic silicone oil b) having at least one phenyl group, preferably chosen from phenyl trimethicone, diphenylsiloxy phenyl trimethicone, and mixtures thereof.

14. The composition according to claim 1, characterised in that the second oil different from the aromatic silicone oil a) is a hydrocarbon oil chosen from hydrocarbon oils of vegetable origin.

15. The composition according to claim 14, characterised in that the hydrocarbon oil of vegetable origin is chosen from jojoba oil, Limnanthes oil, a mixture of caprylic/capric triglycerides, and mixtures thereof.

16. The composition according to claim 15, characterised in that the hydrocarbon oil of vegetable origin is a mixture of caprylic/capric triglycerides.

17. The composition according to claim 1, characterised in that the ratio between the aromatic silicone oil a) and the second oil different from the aromatic silicone oil a) varies from 10/90 to 80/20.

18. The composition according to claim 17, characterised in that the ratio between the aromatic silicone oil a) and the second oil different from the aromatic silicone oil a) varies from 10/90 to 50/50, preferably from 15/85 to 40/60, and more preferentially from to 30/70, when the second oil is an aromatic silicone oil b).

19. The composition according to claim 17, characterised in that the ratio between the aromatic silicone oil a) and the second oil different from the aromatic silicone oil a) varies from 80/20 to 20/80, preferably from 70/30 to 30/70, more preferentially from 65/35 to 35/70, and even more preferentially from 60/40 to 40/60, when the second oil is a hydrocarbon-based oil.

20. The composition according to claim 1, characterised in that the oily phase comprises at least one nacre and/or at least one hydrophobic pigment.

21. The composition according to claim 20, characterised in that the nacre and/or the hydrophobic pigment represents from 0.01% to 5% by weight of the total weight of the composition, and preferably from 0.05% to 2% by weight of the total weight of the composition.

22. The composition according to claim 1, characterised in that it has two non-homogeneous phases, an aqueous phase and an oily phase, which are visually distinct from one another at rest.

23. The composition according to claim 1, characterised in that the oily phase is in the form of bead(s) immiscible within the aqueous phase.

24. The composition according to claim 23, characterised in that oily-phase bead(s) are free of external coating.

25. A use of a cosmetic composition according to claim 1 for skin care and/or as a fragrance.

26. A composition for skin care, characterised in that it comprises a cosmetic composition according to claim 1 and a cosmetic active agent, said cosmetic active agent preferably being retinol palmitate, Lipochroman®-6 (dimethylmethoxy chromanol, CAS number: 83923-51-7), an esterified derivative of vitamin C such as ascorbyl tetraisopalmitate, and more preferentially an esterified derivative of vitamin C such as ascorbyl tetraisopalmitate.

27. A fragrance composition, characterised in that it comprises a cosmetic composition according to claim 1 and a fragrance concentrate.

28. A vial comprising a packaging means and an application means, said packaging means containing the cosmetic composition according to claim 1 and said application means preferably being a sprayer.

29. A skin care and/or fragrance application process characterised in that it comprises the following steps:
i—shaking of a cosmetic composition according to claim 1 for mixing the aqueous phase with the oily phase; and
ii—application of the mixed cosmetic composition obtained at the end of step i—either directly using the fingers or with a cotton wool, or by spraying, and preferably by spraying.
